(19) United States
(12) Patent Application Publication
(10) Pub. No.: US 2004/0151744 A1
(43) Pub. Date: Aug. 5, 2004

(54) Avermectin and praziquantel combination therapy

(75) Inventor: Bernard Frank Bishop, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/761,636

(22) Filed: Jan. 21, 2004

Related U.S. Application Data
(63) Continuation of application No. 10/116,880, filed on Apr. 5, 2002, now abandoned.
(60) Provisional application No. 60/292,395, filed on May 21, 2001.

(51) Int. Cl. A61K 31/7048; A61K 9/00

(52) U.S. Cl. 424/400; 514/29

(57) ABSTRACT

An antiparasitic combination therapy compising a combination of a 13-monosaccharide 5-oxime avermectin, such as selamectin, and praziquantel in a veterinarily acceptable carrier, diluent or adjuvant. Also provided is a method of treatment or prophylaxis of a parasitic infestation as well as a kit useful in the treatment or prophylaxis of a parasitic infestation of flea, heartworm or tapeworm in a mammal.

### BACKGROUND OF THE INVENTION

The present application claims the benefit of U.S. Provisional Patent Application Serial No. 60/292,395, filed May 21, 2001, the contents of which are hereby incorporated by reference.

The invention described herein relates to a combination therapy in the field of treatment and prophylaxis of parasitic infestations and consequences thereof, particularly in veterinary medicine. In particular the invention relates to antiparasitic therapy using a combination of avermectin-like compounds, specifically 13-monosaccharide-5-oxime compounds, and praziquantel.

EP 0 717 993 describes a synergistic combination of avermectins/milbemycins with praziquantel, in particular for the treatment of cestode and nematode infestations in horses. Particular species mentioned therein include Anoplocephala perfiolata, Stiongylidae, Gasterophilus spp., and Parascari aquorum. Particular avermectins mentioned therein include ivermectin, abamectin, moxidectin and doramectin. GB 2252730, EP 0 930 077, WO 98/06407 and WO 95/05181 also describe combinations of avermectins with praziquantel. Selamectin, or 5-oximino-22,23-dihydro-25-cyclohexylayermectin B1, is disclosed as Example 5 in International Patent Publication No. WO94/15944, herein incorporated by reference.

Formulations of selamectin in di(C₂₋₄ glycol) mono(C₁₋₄ alkyl) ethers, including dipropylene glycol monomethyl ether (DPGMME), and alcohols such as ethanol and isopropyl alcohol (IPA) are disclosed in International Patent Publication No. WO00/30449, herein incorporated by reference. Selamectin is the active ingredient of the product marketed under the trade names Revolution™, Stronahold™, etc. by Pfizer Inc. and associated companies, particularly for the treatment (including prophylaxis) of endo- and ecto-parasite infestations in companion animals such as cats and dogs.

Praziquantel is 2-(cyclohexylcarbonyl)-1,2,3,6,7-11b-hexahydro-4H-pyrazino[2,1-a]isoquinolin-4-one, also known as an active ingredient in EMBAY 8440™, Biltricide™, Cesol™, and Droncit™. It is mentioned in the Merck Index, 11th edition, para.7714, and references therein, herein incorporated by reference.

Workers at Pfizer disclosed efficacy data for selamectin vs. gastrointestinal nematodes in cats and ascarids in dogs in Veterinary Parasitology, 91 (2000) 321; 333.

The present inventors have now discovered that a combination of a 13-monosaccharide 5-oxime avermectin derivative, preferably selamectin, with praziquantel, gives good broad spectrum activity vs. endo- and ecto-parasites in companion animals such as dogs and especially cats.

Desirable attributes, in particular for a topical application for administration to companion animals such as cats and dogs, and especially in relation to a treatment for the control. of flea, heartworm and tapeworm infestation, include: efficacy; persistence of efficacy;. low volume; cosmetically acceptable; convenient; need for small number of applications for broad spectrum parasite control; compliant treatment; safe; suitable pharmacokinetic profile; suitable transdermal flux profile; rapid rate of onset; low dose of active ingredient(s); cutaneous tolerability; and stability on storage.

### SUMMARY OF THE INVENTION

Accordingly, the present invention provides a veterinary formulation comprising a 13-monosaccharide 5-oxime avermectin at around 1% to 16% w/v, and praziquantel at around 0.5-10% w/v, in a veterinarily acceptable carrier, diluent or adjuvant.

In addition, the present invention provides a method of treatment and prophylaxis of parasitic infestation of flea, heartworm or tapeworm in a mammal which comprises administering to said mammal an amount of a 13-monosaccharide 5-oxime. avermectin, selected from the group consisting of selamectin, and an amount of praziquantel, each amount being effective to treat or provide prophylaxis to the mammal.

Also provided by the present invention is a kit useful in the treatment or prophylaxis of a parasitic infestation of flea, heartworm or tapeworm in a mammal, which comprises a 13-monosaccharide 5-oxime avermectin, selected from the group consisting of selamectin, and praziquantel and a pharmaceutical or veterinary carrier, and instructions for the treatment of a parasitic infestation of flea, heartworm or tapeworm in a mammal.

### BRIEF DESCRIPTION OF THE DRAWING

FIG. 1 shows that praziquantel solubility is increased as the IPA concentration increases in accord with the present invention.

### DETAILED DESCRIPTION OF THE INVENTION

The present invention demonstrates excellent efficacy against tapeworms (Dipylidium and Taenia spp) for praziquantel dosed topically to cats at a dose of 1 mg/kg, in combination with selamectin at 8 mg/kg in a DPGMME/IPA formulation. Reducing the dose of praziquantel to 0.5 mg/kg reduced efficacy levels against Dipylidium spp. Praziquantel and selamectin loading in the formulation were 1% and 8% respectively, and animals were dosed with a single topical administration between the shoulder blades (0.1 ml/kg). Cats harbouring natural tapeworm infections were used, and randomized to treatment based on pre-trial infections. Animals were killed 30 days after treatment, and post mortem tapeworm counts were conducted.

TABLE A
Summary of topical co-formulation efficacy: selamectin/
praziquantel combinations in cats
Trial “A”

| | | Mean Dipylidium | |
|---|---|---|---|
| | Dose | post mortem | |
| Treatment | (mg/kg) | count (n = 4/gp) | % Efficacy |
| Control | | 12.2 | |
| Selamectin | 8 | 7.2 | 40% |
| Praziquantel | 1 | 3.25 | 74% |
| Sela + Prazi | 8 & 1 | 0.2 | 92% |

Trial “B”

| | | Mean Dipylidium | | Mean Taenia | |
|---|---|---|---|---|---|
| | Dose | post mortem | | post mortem | |
| Treatment | (mg/kg) | count (n = 5/gp) | % Efficacy | count (n = 5/gp) | % Efficacy |
| Control | | 9 | | 4.2 | |
| Selamectin | 8 | No infection | N/A | No infection | N/A |
| Praziquantel | 1 | 1 | 89% | 1.2 | 72% |
| Praziquantel | 0.5 | 7.6 | 16% | 0 | 100% |
| Sela + Prazi | 8 & 1 | 0 | 100% | 0 | 100% |
| Sela + Prazi | 8 & 0.5 | 1.4 | 85% | 0.6 | 86% |

The efficacy of praziquantel against tapeworm infections after oral dosing is summarised in the FOI for Cutter Tape Tabs, an oral tablet approved for use in dogs and cats in the USA. Abstracted information can be found below. In summary, acceptable efficacy was established as 100% elimination of the tapeworm parasites in all animals dosed. Initial oral studies were done with a range of dosages, some as low as 0.5 mg/kg body weight. The results were not always reproducible. and the recommended dosage schedule for cats is a single tablet (23 mg praziquantel) for animals weighing 5-11 pounds (2.3-5.0 kg) (i.e., approx 5-10 mg/kg).

The present topical data indicate that 1 mg/kg praziquantel, in combination with selamectin, will control tapeworm infections.

Praziquantel has a widespread use in animals orally at higher doses than those which we have tested.

Cutter Tape Tabs FOI Abstract

Preclinical Safety Evaluation:

Two cats were treated orally with doses of 25 and 50 mg/kg. There were no signs of clinical toxicity or evidence of gross lesions in the gastrointestinal tract. In another test study, three cats received two oral 100 mg/kg treatments at 14 day intervals in a study. Only nausea and vomiting occurred in two animals with no additional clinical signs observed. No significant clinical pathology or. histopathology changes occurred.

In another test study, seven cats (6 females and 1 male) were treated orally with a 5 mg/kg dose during all critical periods of reproduction. Two groups of seven cats (6 females and 1 male) each were also treated subcutaneously and intramuscularly with. a praziquantel injectable solution with a 5 mg/kg dose during all critical periods of reproduction. The oral use of praziquantel was further evaluated in one male and three female cats in a controlled study. Two groups of 4 cats (1 male and 3 females) each were also treated subcutaneously and intramuscularly with the praziquantel injectable solution. All treated cats received 3× the label rate. Four females and 1 male served as untreated controls.

The treated males received 7 treatments at 2-week intervals throughout the breeding season. Each treated female received a treatment prior to breeding, during the embryogenic period of pregnancy, during late pregnancy and again during lactation. The study confirmed the lack of effects on fertility, conception, fetal development or pregnancy when praziquantel was administered at 3×dosage levels.

Eight cats received three oral doses at 14-day intervals of either 5× or 10× the labeled rate. No significant clinical signs of toxicity were observed, nor did changes occur for hematology, clinical chemistry and histopathology. Two kittens (4½ to 7½ weeks old) were further treated twice at a 14-day interval. The dosage rate was 5× the label dose. Slight depression was observed in one kitten. No significant clinical toxicity signs or clinical pathology and histopathology changes were attributed to this dosage rate.

Clinical Field Trial Safety

The tablet formulation was further administered to 135 cats (eight weeks to 13 years of age and two to 191b) in clinical field trials. One instance of diarrhea and one of salivation (total =2, 1.5%) were reported and were rated as non-significant.

Safety Summary

In summary, the safety index for the use of praziquantel tablets in cats has been derived from controlled studies using the final tablet formulation (vomiting was the only effect with dual treatments of 100 mg/kg at a 14-day interval). Vomiting at high dosage rates is the typical reaction which prevents significant clinical toxicity signs from occurring. The safety factor is at least 5× the label rate when the product is administered at 14 day intervals to kittens 5 1/2 weeks of age and older.

Efficacy of Oral Praziquantel in Cats (FOI Summary)

Indications for Cats

The approved oral product CUTTER Tape-Tabs (praziquantel) Tapeworm Tablets will remove the common tapeworms, Dipylidium caninum and Taenia faeniaeformis, from cats and kittens.

Dosage Form(s), Route(s) of Administration and Recommended Dosage(s):

CUTTER Tape-Tabs (praziquantel) Tapeworm Tablets for Cats and Kittens are sized for easy administration to either adult cats or kittens. The tablets may be given directly in the mouth or crumbled and mixed with the food.

Dosage Schedule for Cats and Kittens'

Cutter Tape-Tabs Tapeworm Tablets

Each tablet contains 23 mg of praziquantel

4 pounds and under ½ tablet

5-11 pounds 1 tablet

Over 11 pounds 1½ tablets

*Not intended for use in kittens less than 6 weeks of age.

Effectiveness:

Studies were conducted to determine the dosage and formulation of praziquantel which produced the most reliable results when used for the removal of tapeworms from cats.

Acceptable efficacy was established as 100% elimination of the tapeworm parasites in all animals dosed. Initial oral studies were done with a range of dosages, some as low as 0.5 mg/kg body weight. The results were not always reproducible.

Twenty-five separate well-controlled critical anthelmintic studies (which involves the sacrificing of animals and examination to determine the number of parasites in the intestinal tract) were conducted with the final tablet formulation in dogs and cats.

A summary table of the investigators' results appears below. Two hundred and ninety-two animals were studied; 157 (100 dogs and 57 cats) were treated with praziquantel tablets orally or in food and 135 (80 dogs and 55 cats) served as untreated controls. Both natural and experimental infections were studied with some animals being infected with two species of tapeworms. All dogs and cats treated according to the recommended dosage schedule, and some treated at less than the recommended dosage schedule, were cleared of their tapeworm infections. At the same time, the untreated control animals, confirmed as positive before treatment, maintained their tapeworm infections, with. the exception of four dogs and one cat that lost their infections spontaneously. In these studies, praziquantel tablets were 100% effective in the treatment of tapeworm infections of dogs and cats due to Taenia pisiformis, Taenia taeniaeformis and Dipylidium caninum. Additionally praziquantel effectively (100%) eliminated experimental T. taeniaeformis infections as young as seven (7) days from cats.

TABLE B
Summary of Preclinical Effectiveness Data for Praziquantel Tablets
in Cats

| | |
|---|---|
| Treated | % Efficacy Against Tapeworms |

| | Cats | T. taeniaeformis | D. caninum |
|---|---|---|---|
| | 4 | Not Studied | 100 |
| | 24 | 100 | 100 |
| | 29* | 100** | 100 |
| TOTAL | 57 | | |

*One animal underdosed was not cleared of its infection.
**Includes cats with experimentally induced immature (7-day old) infections.

Field investigations of praziquantel tablets were conducted. The field trials were well-controlled using bunamidine hydrochloride as the positive control drug. Overall, 279 dogs and 173 cats were studied, including a wide range of ages, breeds, and weights of both sexes. Praziquantel tablets were administered to 218 dogs and 135 cats while 61 dogs and 38 cats were dosed with bunamidine hydrochloride. Dosing was administered according to label directions. The animals were observed for the presence of tapeworms proglottids 10-14 days post-treatment; any proglottid found was identified. Investigators were asked to evaluate praziquantel for ease of administration, efficacy and safety on a scale of excellent, good, fair and poor. Investigators rated efficacy in dogs as excellent to good in 97% of the cases in dogs and cats. These trials confirmed preclinical efficacy results and demonstrated that praziquantel tablets, when used according to label directions, did have the effect it purports in its labeling.

Formulation

A range of solubility studies have shown that in any ratio of IPA/DPGMME, praziquantel has a solubility above 60 mg/g (6% w/w) even in the presence of selamectin at up to 120 mg/g (12% w/w). Above 50% IPA however, this solubility may be increased to over 90 mg/g (9% w/w) in the supersaturated state. These data indicate that a formulation of selamectin and praziquantel in combination, with levels of up to 9% w/w praziquantel /12% w/w selamectin would be possible in IPA/DPGMME mixtures.

As part of a feasibility study into the development of a selamectin/praziquantel combination topical formulation, the solubility of praziquantel in the IPA/DPGMME type vehicle used for development of Selamectin Topical Solution (Stronghold™/Revolution™), was determined.

To achieve this a series of solutions of varying IPA/DPGMME ratio were prepared. To help minimize any solubility issues, which could arise due to the presence of selamectin in a formulation, a constant amount of selamectin was added to each solution. After achieving saturated solubility in these solutions and following filtration to remove excess undissolved praziquantel, the level of praziquantel (and selamectin) in solution was determined by HPLC.

Preparation of Solutions

A series of IPA/DPGMME mixtures were prepared in duplicate as outlined in Table 1. To these were added a fixed amount of selamectin to provide a constant concentration of 120 mg per ml, i.e., the maximum selamectin concentration found in Stronghold™. After preparation these solutions were placed at 30° C. and rolled continuously for 48 hours. On inspection it was found that complete dissolution of the praziquantel had occurred and so further praziquantel was added until a saturated solution was obtained.

TABLE 1
SOLVENT MIXTURES FOR PRAZIQUANTEL SOLUBILITY
STUDIES

| | IPA | DPGMME |
|---|---|---|
| Solution | % V/V | % V/V |
| 1A | 100 | 0 |
| 1B | 100 | 0 |
| 2A | 75 | 25 |
| 2B^{[1]} | 75 | 25 |
| 3A | 50 | 50 |
| 3B | 50 | 50 |
| 4A | 25 | 75 |
| 4B | 25 | 75 |
| 5A | 0 | 100 |
| 5B | 0 | 100 |

^{[1]}Solution 2B was broken during preparation and so only a single solution (2A) was assayed.

Praziquantel Analysis

Praziquantel was analysed using an gradient HPLC method modified from that developed for selamectin. This method was validated with respect to praziquantel linearity over the range 10 to 200 mg per ml of praziquantel (in the solubility test solutions) and in the presence of 120 mg per ml of selamectin. This same method was also used to check on the selamectin content in these same solutions.

Results

The solubility of praziquantel and selamectin in the IPA/DPGMME solutions was calculated as both mg per g and mg per ml. The mass of a 1 ml solution taken for analysis was also measured so that an approximate density could be calculated. This data is shown in Table 2 and the mean values are shown graphically in the Figure below.

TABLE 2
SOLUBILITY RESULTS FOR PRAZIQUANTEL AND
SELAMECTIN IN IPA/DPGMME SOLUTIONS

| | Concentration (mg per ml) | | Density | Concentration (mg per g) | |
|---|---|---|---|---|---|
| Solution | Praziquantel | Selamectin | g/ml | Praziquantel | Selamectin |
| 1A | 87.1 | 103 | 0.84 | 103.7 | 123 |
| 1B | 84.4 | 103 | 0.84 | 100.5 | 123 |
| 2A | 80.2 | 103 | 0.87 | 92.2 | 118 |
| 3A | 81.7 | 103 | 0.91 | 89.8 | 113 |
| 3B | 82.2 | 103 | 0.91 | 90.3 | 113 |
| 4A | 74.9 | 107 | 0.95 | 78.8 | 113 |
| 4B | 74.9 | 103 | 0.95 | 78.8 | 108 |
| 5A | 65.6 | 103 | 0.99 | 66.3 | 104 |
| 5B | 68.2 | 102 | 0.99 | 68.9 | 103 |

For both praziquantel and selamectin there is a relationship between % IPA in the solvent mixture and increasing solubility, although this is much less marked for selamectin. If the solubility data are taken solely on a mg/ml basis then this trend is still present but it is much less visible. The reasons for this are two fold. Firstly as the solvent mixture changes from IPA to DPGMME the density of the solution increases (given that the densities of IPA and DPGMME are 0.787 and 0.950 g/cm³ respectively) and secondly as the amount of praziquantel dissolved in solution increases, so does the total amount of solids and so this leads to an increase in density. Given that solubility increases with increasing IPA, then these two effects work counter to each other. A better guide to solubility effects is therefore found by using the mass/mass data.

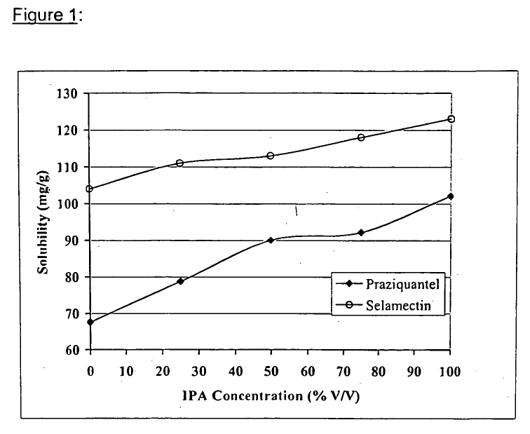

The data in table 2 and shown graphically in FIG. 1, both indicate that praziquantel solubility is increased as the IPA concentration increases. The range of praziquantel solubilities found (based on mean values) are between 68 mg/g (67 mg/ml) in 100% DPGMME, increasing to 102 mg/g (86 mg/ml) for a 100% IPA solution. It should be noted that above 50% IPA the solubility of praziquantel changes relatively little increasing IPA level and is always above the 90 mg/g level. There is also a decrease in selamectin solubility as the IPA concentration reduces although this never goes below 100 mg/g. In fact this process is used in selamectin topical solution to create a selamectin saturated DPGMME solution as the IPA evaporates on the back of the treated animals and so drive the selamectin through the dermal layer.

### EXAMPLE FORMULATIONS

Experimental data shows, that in a solvent mixture of IPA/DPGME, varying from 0 to 100% v/v IPA, praziquantel can be solubilised at concentrations of up to 10% w/w in the presence of up to 12% w/w selamectin. Given the requirement to ensure that both selamectin and praziquantel remain solubilised after evaporation of the IPA. and to minimise the risk of selamectin and praziquantel precipitation from the formulation on storage, an Example formulation is:

### Example 1

| Ingredient | Composition (% w/v) | Function |
|---|---|---|
| Selamectin | 6.0 (note a) | Active ingredient |
| Praziquantel | 6.0 (note b) | Active ingredient |
| BHT | 0.08 | Antioxidant |
| DPGMME | 5.6 | Solubiliser |
| IPA | to volume (note c) | Solubiliser |

Notes:
(a) Assumes 100% potency - the actual amount used is adjusted according to assay value
(b) Assumes 100% potency - the actual amount used is adjusted according to assay value
(c) The quantity of IPA is adjusted to correct for the amounts of selamectin and praziquantel used.

The formulation as detailed in Examples 1-5 can be prepared by the following method:—

1) Add the appropriate amount of IPA to mixing vessel.

2) Add the appropriate amount of DPGMME to mixing vessel.

3) Mix the two solvents together until homogeneous.

4) Add the BHT to the solution and mix until dissolved.

5) Add the appropriate amount selamectin to the solution.

6) Add the appropriate amount of praziquantel to the solution.

7) Mix until all the selamectin and praziquantel have dissolved.

Further Examples of formulations containing selamectin and praziquantel are shown below, wherein the composition within the formulation is expressed % weight by volume:

### Example 2

| Ingredient | Composition (% w/v) | Function |
|---|---|---|
| Selamectin | 8.0 (note a) | Active ingredient |
| Praziquantel | 0.5 (note b) | Active ingredient |
| BHT | 0.08 | Antioxidant |
| DPGMME | 5.6 | Solubiliser |
| IPA | to volume (note c) | Solubiliser |

### Example 3

| Ingredient | Composition (% w/v) | Function |
|---|---|---|
| Selamectin | 8.0 (note a) | Active ingredient |
| Praziquantel | 1.0 (note b) | Active ingredient |
| BHT | 0.08 | Antioxidant |
| DPGMME | 5.6 | Solubiliser |
| IPA | to volume (note c) | Solubiliser |

### Example 4

| Ingredient | Composition (% w/v) | Function |
|---|---|---|
| Selamectin | 12.0 (note a) | Active ingredient |
| Praziquantel | 9.0 (note b) | Active ingredient |
| BHT | 0.08 | Antioxidant |
| DPGMME | 5.6 | Solubiliser |
| IPA | to volume (note c) | Solubiliser |

### Example 5

| Ingredient | Composition (% w/v) | Function |
|---|---|---|
| Selamectin | 6.0 (note a) | Active ingredient |
| Praziquantel | 3.0 (note b) | Active ingredient |
| BHT | 0.08 | Antioxidant |
| DPGMME | 5.6 | Solubiliser |
| IPA | to volume (note c) | Solubiliser |

Notes:
(a) Assumes 100% potency - the actual amount used is adjusted according to assay value
(b) Assumes 100% potency - the actual amount used is adjusted according to assay value
(c) The quantity of IPA is adjusted to correct for the amounts of setamectin and praziquantel used.

An aspect of the invention is the provision of a combination therapy using a formulation comprising a 13-monosaccharide 5-oxime avermectin such as selamectin at around 6-12% w/v, and praziquantel at around 3-9% w/v (preferably around 6% w/v), in a veterinarily acceptable carrier, diluent or adjuvant.

Preferably the (13-monosaccharide 5-oxime avermectin such as selamectin)-containing formulation comprises a di(C₂₋₄ glycol) mono(C₁₋₄ alkyl) ether and an optional skin-acceptable solvent.

Preferably the (13-monosaccharide 5-oxime avermectin such as selamectin)-containing formulation is suitable for topical, preferably spot-on, application.

Another aspect of the invention is the provision of an antiparasitic combination therapy whereby a 13-monosaccharide 5-oxime avermectin such as selamectin is provided at around 6-12 mg/kg (re. host animal) and praziquantel is provided at up to 18 mg/kg (re. host animal). Preferably the selamectin is present in the formulation at about 1% to about 16% (w/v), more preferably about 4% to about 12% w/v. and most preferably about 6% to about 12% w/v. Specific preferred formulations contain selamectin at about 6% w/v and about 12% w/v.

Preferably the praziquantelis present in the formulation at about 0.5 to about 10% w/v, more preferably about 3 to about 9% w/v , most preferably about 6% w/v.

Preferably the di(C₂₋₄ glycol) mono(C₁₋₄ alkyl) ether is diethylene glycol monomethyl ether (DEGMME) or dipropylene glycol monomethyl ether (DPGMME), more preferably DPGMME.

Preferably the skin-acceptable solvent is present and is ethanol or isopropanol, more preferably isopropanol.

Preferably the formulation containing the avermectin 13-monosaccharide oxime has a w/v to v/v ratio of avermectin 13-monosaccharide oxime to the glycol monomethyl ether is in the range (0.5 to 2) to 1, more preferably (0.7 to 1.4) to 1, yet more preferably (0.9 to 1.1) to 1, most preferably about 1:1.

A preferred formulation according to the invention also contains antioxidant, preferably selected from propylgallate, BHA (2-t-butyl-4-methoxyphenol), and BHT (2,6-di-t-butyl-4-methylphenol), more preferably BHT.

A preferred formulation according to the invention consists of:.

(a) selamectin, at a level of 1% to 16% w/v;

(b) DEGMME or DPGMME at 1. to 16% w/v, and at a w/v or v/v ratio of active compound to DEGMME/DPGMME of about 1:1;

(c) praziquantel at 0.5 to 10% w/v;

(d) isopropanol to volume (100%);

and, optionally (e) BHT (at less than 0.1% w/v).

A more preferred formulation consists of

(a) selamectin at a level of 6% to 12% w/v;

(b) DEGMME or DPGMME 6 to 12% w/v , and at a w/v or v/v ratio of active compound to DEGMME/DPGMME of about 1:1;

(c) praziquantel at 3-9% w/v;

(d) isopropanol to volume (100%);

and, optionally (e) BHT (at less than 0.1% w/v).

The most preferable formulations are those described in the Examples, especially Example 1.

Further aspects of the invention include:

(a) a method of treatment (including prophylaxis) of parasitic infestation of flea and/or heartworm and tapeworm in a mammal which comprises treatment with a 13-monosaccharide 5-oxime avermectin, such as selamectin, and praziquantel;

(b) a method of treatment as in (a) wherein the 13-monosaccharide 5-oxime avermectin such as selamectin and praziquantel are administered separately in different formulations;

(c) a method of treatment as in (a) wherein the 13-monosaccharide 5-oxime avermectin such as selamectin and praziquantel are administered in the same formulation;

(d) a method of treatment as in (a) or (b) wherein the 13-monosaccharide 5-oxime avermectin such as selamectin and praziquantel are administered via the same route;

(e) a method of treatment as in (a) or (b) wherein the 13-monosaccharide 5-oxime avermectin such as selamectin and praziquantel are administered via different routes;

(f) a pharmaceutical or veterinary composition which comprises a 13-monosaccharide 5-oxime avermectin such as selamectin and praziquantel and a pharmaceutical or veterinary carrier;

(g) the use of selamectin and praziquantel in the manufacture of a medicament for the treatment of a parasitic infestation, or condition mediated by a parasitic infestation, on or in a mammal. especially a companion animal such as a cat;

(g) a kit useful in the treatment of a parasitic infestation of flea and/or heartworm and tapeworm in a mammal, which comprises a 13-monosaccharide 5-oxime avermectin, such as selamectin, and praziquantel and a pharmaceutical or veterinary carrier, and instructions for the treatment of a parasitic infestation of flea and/or heartworm and tapeworm in a mammal.

What is claimed is:

1. A veterinary formulation comprising a 13-monosaccharide 5-oxime avermectin at around 1% to 16% w/v, and praziquantel at around 0.5-10% w/v, in a veterinarily acceptable carrier, diluent or adjuvant.

2. The formulation according to claim 1 wherein the 13-monosaccharide 5-oxime avermectin is selamectin.

3. The formulation according to claim 1 wherein the praziquantel is present at around 6% w/v.

4. The formulation according to claim 1 which further comprises a di(C₂4 glycol) mono(C₁₋₄ alkyl) ether and an optional skin-acceptable solvent.

5. The formulation according to claim 1 which is suitable for topical application or spot-on application.

6. The formulation according to claim 1 wherein the 13-monosaccharide 5-oxime avermectin is provided at around 6-12 mg/kg (with respect to the host animal) and praziquantel is provided at up to 18 mg/kg (with respect to the host animal).

7. The formulation according to claim 1 wherein the 13-monosaccharide 5-oxime avermectin is present at about 6% to about 12% w/v.

8. The formulation according to claim 1 wherein the praziquantel is present in the formulation at about 3% to about 9% w/v.

9. The formulation according to claim 1 wherein the praziquantel is present in the formulation at about 6% w/v.

10. The formulation according to claim 1 wherein a di(C₂₋₄ glycol) mono(C₁₋₄ alkyl) ether is present, said ether being diethylene glycol monomethyl ether (DEGMME) or dipropylene glycol monomethyl ether (DPGMME).

11. The formulation according to claim 1 wherein a skin-acceptable solvent is present, said solvent being ethanol or isopropanol.

12. The formulation according to claim 1 which consists of:
(a) selamectin, at a level of 1% to 16% w/v;
(b) DEGMME or DPGMME at 1 to 16% w/v, and at a w/v or v/v ratio of active compound to DEGMME/DPGMME of about 1:1;
(c) praziquantel at 0.5 to 10% w/v;
(d) isopropanol to volume (100%);
and, optionally (e) BHT (at less than 0.1% w/v).

13. A method of treatment and prophylaxis of parasitic infestation of flea, heartworm or tapeworm in a mammal which comprises administering to said mammal an amount of a 13-monosaccharide 5-oxime avermectin, selected from the group consisting of selamectin, and an amount of praziquantel, each amount being effective to treat or provide prophylaxis to the mammal.

14. The method according to claim 13 wherein the 13-monosaccharide 5-oxime avermectin, selected from the group consisting of selamectin, and praziquantel are administered separately in different formulations.

15. The method according to claim 13 wherein the 13-monosaccharide 5-oxime avermectin, selected from the group consisting of selamectin, and praziquantel are administered in the same formulation.

16. The method according to claim 13 wherein the 13-monosaccharide 5-oxime avermectin, selected from the group consisting of selamectin, and praziquantel are administered via the same route.

17. The method according to claim 13 wherein the 13-monosaccharide 5-oxime avermectin, selected from the group consisting of selamectin, and praziquantel are administered via different routes.

18. The method according to claim 13 wherein the mammal is a companion animal, selected from the group consisting of a cat.

19. A kit useful in the treatment or prophylaxis of a parasitic infestation of flea, heartworm or tapeworm in a mammal, which comprises a 13-monosaccharide 5-oxime avermectin, selected from the group consisting of selamectin, and praziquantel and a pharmaceutical or veterinary carrier, and instructions for the treatment of a parasitic infestation of flea, heartworm or tapeworm in a mammal.
